(19) United States
(12) Patent Application Publication
(10) Pub. No.: US 2026/0053601 A1
(43) Pub. Date: Feb. 26, 2026

(54) METHOD TO ESTIMATE THE THREE-DIMENSIONAL STRUCTURE OF TEETH

(71) Applicant: ZAAMIGO AG, Zurich (CH)

(72) Inventors: Severin STALDER, Zurich (CH); Riccardo BARAVELLI, Lugano (CH)

(73) Assignee: ZAAMIGO AG, Zurich (CH)

(21) Appl. No.: 19/108,489

(22) PCT Filed: Jun. 27, 2023
(86) PCT No.: PCT/EP2023/067485

§ 371 Date: Mar. 4, 2025

Related U.S. Application Data
(60) Provisional application No. 63/403,827, filed on Sep. 5, 2022.

(51) Int. Cl. A61C 9/00; G06T 7/00; G06T 17/00

(52) U.S. Cl. CPC A61C 9/0053; G06T 7/0014; G06T 17/00; G06T 2207/10024; G06T 2207/30036; G06T 2210/41; G06T 2210/56

(57) ABSTRACT

Provided herein are methods, systems, algorithms, computer programs, kits, devices, and computer-executable code for generating a three-dimensional (3D) representation of a user's tooth or teeth from two-dimensional (2D) images.

### CROSS-REFERENCE

This application claims the benefit of U.S. Provisional Application No. 63/403,827, filed Sep. 5, 2022, which application is incorporated herein by reference in its entirety.

### BACKGROUND

Obtaining 3D dental features and capturing a replica of the mouth through methods such as dental impressions or intraoral scanning scans are a necessary part of dentistry. 3D dental features can aid a dentist in diagnosis and devising treatment plans. Obtaining such features are also necessary for manufacturing of dental aligner, braces, and dental prosthetics.

### SUMMARY

In some embodiments, the present disclosure provides a device comprising: an elongated body comprising: a) a head end comprising a face; b) a tail end comprising a wireless connection to a power supply; and c) a shaft connecting the head end and the tail end, wherein the shaft comprises a shaft central axis through the shaft from the head end to the tail end, wherein the face comprises: 1) an optical sensor having a field of view, wherein the optical sensor has a central optical axis that is perpendicular to the field of view, wherein the central optical axis is perpendicular to the shaft central axis, wherein the optical sensor is configured to capture an image; and 2) a plurality of sources of illumination oriented to illuminate the field of view.

In some embodiments, the present disclosure provides a method comprising a) obtaining by an optical sensor a set of two-dimensional images of a tooth; and b) generating a three-dimensional representation of the tooth based at least in part on the set of two-dimensional images of the tooth.

In some embodiments, the present disclosure provides a kit comprising a) the device described herein; and b) written instructions describing a use of the device in scanning a tooth.

In some embodiments, the present disclosure provides a kit comprising a) the device described herein; and b) written instructions describing a use of the device in the method described herein.

### BRIEF DESCRIPTION OF THE DRAWINGS

FIG. 1 illustrates a schematic of a system for generating a 3D representation of a subject's tooth from 2D images.

FIG. 2 illustrates the head end comprising an intra-oral camera or camera system for obtaining image(s) of a tooth described herein.

FIG. 3 depicts an intra-oral camera or camera system described herein.

FIG. 4 depicts an embodiment of a 2D image acquired with the method described herein.

FIG. 5 illustrates an embodiment of a 2D image in pixels.

FIG. 6 illustrates an exemplary schematic of a workflow for the trained, machine-learning model described herein.

### DETAILED DESCRIPTION

Obtaining accurate three-dimensional (3D) representation dental features is necessary for manufacturing dental aligner, braces, and dental prosthetics. Dental impressions for generating such a 3D model can be taken by a user or an orthodontic professional using a dental impression kit. An intraoral scan of the user's mouth can be taken using a 3D scanner. However, these methodologies for obtaining information necessary to generate a 3D model of the user's teeth can be time consuming, prone to errors made by the user, and require specialized equipment.

3D scanners often rely on sophisticated sensors, such as a high-speed optical sensor with a global shutter. These sensors often include a pattern projector and depend on an electrical motor that drives part of the optics. These sensors also comprise of high-grade optics. The complicated design of these 3D scanners often results in a bulky apparatus that limits the accessibility to all areas of the mouth. Scanning of up to 50,000 images is often necessary during a scan using these 3D scanners. Such extensive scanning requires high data bandwidth for data transmission. These scanners often require frequent, time-consuming calibration between use. As such, the high equipment and labor/skills costs required for this technique limits the utility.

The disclosure provides methods, systems, algorithms, computer programs, kits, devices, and computer-executable code for generating a three-dimensional (3D) representation of a user's dental features from two-dimensional (2D) images. The methods, systems, algorithms, computer programs, kits, devices, and computer-executable code can be used to generate a 3D representation of a user's dental features from 2D images taken by an intra-oral camera or camera system. The methods, systems, algorithms, computer programs, kits, devices, and computer-executable code can generate a 3D representation of a user's dental features using a trained, machine-learning model.

In some embodiments, the 3D representation is characterized by accuracy. Accuracy can refer to the closeness of agreement between a test result and the accepted reference value. For example, the test result can denote an observed, calculated or estimated value and the accepted reference value can denote the true value. In some embodiments, the test result is the 3D representation and reference value is the ground truth for the measured tooth or teeth.

In some embodiments, the accuracy of the 3D representation is about 1-1000 μm, characterized by high precision and high trueness. In some embodiments, the accuracy is measured as the max point distance between the test result and the reference value.

In some embodiments, the methods, systems, algorithms, computer programs, kits, devices, and computer-executable code described herein can generate 3D representation of a tooth or teeth for preparation of dental objects such as dental aligners, braces, prosthetics, hardware, or appliances, either directly or through a variety of intermediate processing steps. In some embodiments, surface data are acquired from a dental model such as a dental prosthetic to ensure proper fitting using a previous scan of corresponding dentition, such as a tooth surface prepared for the prosthetic.

In some embodiments, the methods, systems, algorithms, computer programs, kits, devices, and computer-executable code described herein can be used to generate 3D representation of a user's tooth or teeth from 2D images by using a trained, machine-learning model comprising a training set comprising reference 2D images of a tooth or teeth with pre-determined 3D structures of the reference tooth or teeth. In some embodiments, the reference tooth or teeth are a real tooth or teeth. In some embodiments, the reference tooth or teeth are synthetically generated tooth or teeth. In some embodiments, the synthetically generated tooth or teeth is generated from a cast. In some embodiments, the synthetically generated tooth or teeth is generated virtually.

In some embodiments, the methods, systems, algorithms, computer programs, kits, devices, and computer-executable code described herein allow for the generation of 3D dental representation using a single optical sensor. In some embodiments, the single optical sensor does not need to be calibrated prior to use

In some embodiments, the methods, systems, algorithms, computer programs, kits, devices, and computer-executable code described herein described herein include more than one optical sensors.

In some embodiments, the methods, systems, algorithms, computer programs, kits, devices, and computer-executable code described herein described herein allow generation of 3D dental representation using a single image. In some embodiments, devices described herein generates a 3D dental representation using more than one image, for example, three images.

### Model Generation System

In some embodiments, the methods, systems, algorithms, computer programs, kits, devices, and computer-executable code described herein can be used to generate a 3D dental representation in a subject in need thereof. The subject can include an adult or a child.

In some embodiments, the disclosure provides a device comprising: an elongated body comprising: a) a head end comprising a face: b) a tail end comprising a wireless connection to a power supply; and c) a shaft connecting the head end and the tail end, wherein the shaft comprises a shaft central axis through the shaft from the head end to the tail end, wherein the face comprises: 1) a first optical sensor having a first field of view, wherein the first optical sensor has a first central axis that is perpendicular to the first field of view, wherein the first central axis is perpendicular to the shaft central axis; 2) a second optical sensor having a second field of view, wherein the second optical sensor has a second central axis that is perpendicular to the second field of view, wherein the second central axis is perpendicular to the shaft central axis, wherein the second central axis is parallel to the first central axis, wherein the first field of view and the second field of view overlap; and 3) a plurality of sources of illumination oriented to illuminate the first field of view and the second field of view.

In some embodiments, the first optical sensor is configured to take a first two-dimensional image, and the second optical sensor is configured to take a second two-dimensional image. In some embodiments, the first two-dimensional image and the second two-dimensional image do not overlap. In some embodiments, the first two-dimensional image and the second two-dimensional image partially overlap and are not coterminous. In some embodiments, the first optical sensor and the second optical sensor have the same focal length. In some embodiments, the first optical sensor and the second optical sensor have different focal length. In some embodiments, the focal length of the first optical sensor is greater than the focal length of the second optical sensor. In some embodiments, the focal length of the second optical sensor is greater than the focal length of the first optical sensor.

In some embodiments, a computational unit generates a three-dimensional representation of a tooth based at least in part on an image of the tooth obtained by the first optical sensor and an image of the tooth obtained by the second optical sensor. In some embodiments, the computational unit generates a three-dimensional representation of a tooth based at least in part by comparing an image of the tooth obtained by the first optical sensor and an image of the tooth obtained by the second optical sensor to a plurality of training images, wherein each training image is associated with a pre-determined three-dimensional structure. In some embodiments, the device described herein can be an imaging device. In some embodiments, the device described herein can be an intra-oral camera.

In some embodiments, the method described herein comprises: a) exposing a tooth to an optical sensor; b) obtaining by the optical sensor a set of two-dimensional images of a tooth; and c) generating a three-dimensional representation of the tooth based at least in part on the set of two-dimensional images of the tooth.

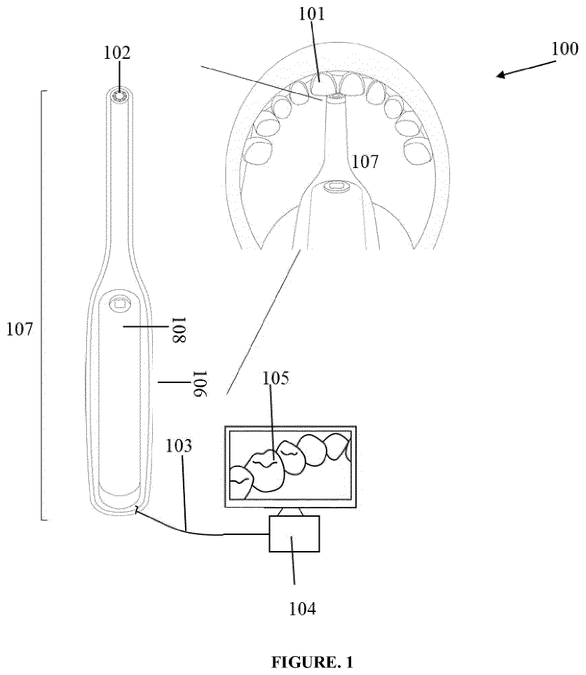

FIG. 1 illustrates a schematic of a system 100 for generating a 3D representation of a subject's tooth or teeth from 2D images. The system 100 comprises a device 107, a computational unit 104, and a display unit 105.

The device 107 has an elongated body comprising a head end 102 comprising a face; a tail end 103 comprising a wireless connection to a power supply; and a shaft 106 connecting the head and the tail end. In some embodiments, the shaft comprises a shaft central axis through the shaft from the head end to the tail end.

The head end 102 comprising a face comprises an intra-oral camera that captures 2D images of the subject's tooth or teeth 101. The tail end 103 comprises a connection to the computational unit 104. The connection can establish communication between the device 107 and the computational unit 104.

The elongated body of the device 107 can have a contour 108, wherein the contour provides a bulge between the head end 102 and the tail end 103. The bulge can comprise a grip. The contour 108 can have at least one user input device, such as a button, lever, dial, thumb wheel, or switch for user control of the system 100 such as starting and stopping the image acquisition process. Non-limiting examples of the user input device include a start scan control, a pause control, a stop control, a clear control, a save control, a retrieve control, and an illumination control. The face comprising the intra-oral camera can be located at the head end 102 of the probe and the user input device can be located at the tail end 103 of the probe.

In some embodiments, the shaft 106 can be a handheld, freely positionable probe.

In some embodiments, the bulge has a bulge greatest width, the head end has a head greatest width, and the tail end has a tail greatest width, wherein the bulge greatest width is greater than the head greatest width, and wherein the bulge greatest width is greater than the tail greatest width.

In some embodiments, the subject's dental feature 101 comprises a tooth, multiple teeth, a portion of a tooth, a structure of a tooth, a maxillary dental arch, a mandibular dental arch, one or more features of a tooth, or any structure typically found in the mouth. In some embodiments, the structure comprises the gum, the tongue, or the cheek.

In some embodiments, the device 107 comprises a distance holder. In some embodiments, the distance holder assists the user to take images with the device 107 while maintaining a fixed distance from the teeth so that the head end 102 can capture sharp pictures.

In some embodiments, the device 107 comprises a mirror. In some embodiments, the mirror is located at the head end 102.

The head end 102 comprises a face comprising an intra-oral camera. The intra-oral camera can capture a set of 2D images of the subject's tooth or teeth during an image acquisition process. In some embodiments, the intra-oral camera acquires the set of 2D images in a continuous process. In some embodiments, the intra-oral camera acquires the set of 2D images in a discrete process.

During the image acquisition process, the intra-oral camera captures the set of 2D images having sufficient spatial resolution and accuracy to generate a 3D model.

In some embodiments, the set of 2D images comprises 2D images of a single tooth.

In some embodiments, the set of 2D images comprises at least 3, at least 4, at least 5, at least 6, at least 7, at least 8, at least 9, or at least 10 2D images of the tooth.

In some embodiments, the set of 2D images comprises at most 3, at most 4, at most 5, at most 6, at most 7, at most 8, at most 9, or at most 10 2D images of the tooth.

In some embodiments, the set 2D images per tooth are taken from the occlusal view, mesial view, distal view, buccal view, lingual view, or a combination thereof of the tooth.

In some embodiments, the set of 2D images comprises at most 1, at most 2, at most 3, at most 4, or at most 5 occlusal images of the tooth.

In some embodiments, the set of 2D images comprises at most 1, at most 2, at most 3, at most 4, or at most 5 mesial images of the tooth.

In some embodiments, the set of 2D images comprises at most 1, at most 2, at most 3, at most 4, or at most 5 distal images of the tooth.

In some embodiments, the set of 2D images comprises at most 1, at most 2, at most 3, at most 4, or at most 5 buccal images of the tooth.

In some embodiments, the set of 2D images comprises at most 1, at most 2, at most 3, at most 4, or at most 5 lingual images of the tooth.

In some embodiments, each 2D image in the set of 2D images overlaps with at least one other 2D image.

In some embodiments, each 2D image in the set of 2D images overlaps with at least 1, at least 2, at least 3, at least 4, or at least 5 other 2D images.

In some embodiments, the overlap can range from about 10% to about 90%. For examples, the overlap can be at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%.

Non-limiting examples of the overlap include tooth size, tooth shape, tooth location, tooth orientation, crown size, crown shape, gingiva location, gingiva shape or contours, tooth-to-gingiva interface location, and interproximal region location.

In some embodiments, the set of 2D images comprises 2D images of more than one tooth. In some embodiments, the set of 2D images comprises 2D images from 2 teeth, 3 teeth, 4 teeth, 5 teeth, 6 teeth, 7 teeth, 8 teeth, 9 teeth, 10 teeth, 11 teeth, 12 teeth, 13 teeth, 14 teeth, 15 teeth, 16 teeth, 17 teeth, 18 teeth, 19 teeth, 20 teeth, 21 teeth, 22 teeth, 23 teeth, 24 teeth, 25 teeth, 26 teeth, 27 teeth, 28 teeth, 29 teeth, 30 teeth, 31 teeth, or 32 teeth.

In some embodiments, the set of 2D images comprising more than one tooth can range from about 3 to about 10,000 or more 2D images. For example, the set of 2D images can comprise about 1, about 2, about 3, about 4, about 5, about 6, about 7, about 8, about 9, about 10, about 60, about 110, about 160, about 210, about 260, about 310, about 360, about 410, about 460, about 510, about 560, about 610, about 660, about 710, about 760, about 810, about 860, about 910, about 960, about 1010, about 1060, about 1110, about 1160, about 1210, about 1260, about 1310, about 1360, about 1410, about 1460, about 1510, about 1560, about 1610, about 1660, about 1710, about 1760, about 1810, about 1860, about 1910, about 1960, about 2010, about 2060, about 2110, about 2160, about 2210, about 2260, about 2310, about 2360, about 2410, about 2460, about 2510, about 2560, about 2610, about 2660, about 2710, about 2760, about 2810, about 2860, about 2910, about 2960, about 3010, about 3060, about 3110, about 3160, about 3210, about 3260, about 3310, about 3360, about 3410, about 3460, about 3510, about 3560, about 3610, about 3660, about 3710, about 3760, about 3810, about 3860, about 3910, about 3960, about 4010, about 4060, about 4110, about 4160, about 4210, about 4260, about 4310, about 4360, about 4410, about 4460, about 4510, about 4560, about 4610, about 4660, about 4710, about 4760, about 4810, about 4860, about 4910, about 4960, about 5010, about 5060, about 5110, about 5160, about 5210, about 5260, about 5310, about 5360, about 5410, about 5460, about 5510, about 5560, about 5610, about 5660, about 5710, about 5760, about 5810, about 5860, about 5910, about 5960, about 6010, about 6060, about 6110, about 6160, about 6210, about 6260, about 6310, about 6360, about 6410, about 6460, about 6510, about 6560, about 6610, about 6660, about 6710, about 6760, about 6810, about 6860, about 6910, about 6960, about 7010, about 7060, about 7110, about 7160, about 7210, about 7260, about 7310, about 7360, about 7410, about 7460, about 7510, about 7560, about 7610, about 7660, about 7710, about 7760, about 7810, about 7860, about 7910, about 7960, about 8010, about 8060, about 8110, about 8160, about 8210, about 8260, about 8310, about 8360, about 8410, about 8460, about 8510, about 8560, about 8610, about 8660, about 8710, about 8760, about 8810, about 8860, about 8910, about 8960, about 9010, about 9060, about 9110, about 9160, about 9210, about 9260, about 9310, about 9360, about 9410, about 9460, about 9510, about 9560, about 9610, about 9660, about 9710, about 9760, about 9810, about 9860, about 9910, about 9960, about 10,000 2D images of more than one tooth.

In some embodiments, the set of 2D images comprises 2D images of the same tooth.

In some embodiments, the set of 2D images comprises 2D images of different teeth.

In some embodiments, obtaining a set of 2D images by the optical sensor can take between 5 seconds to 15 minutes. For example, obtaining a set of 2D images by the optical sensor can take about 5 seconds, about 10 seconds, about 15 seconds, about 20 seconds, about 25 seconds, about 30 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, or about 15 minutes.

In some embodiments, a user can be trained to perform the image acquisition process. The frequency of performing the image acquisition process by the user can impact the duration of the image acquisition process. For example, the duration of the image acquisition process can be decreased when a user frequently performs the process.

In some embodiments, the device 107 can have a dimension of about 210 mm×about 26 mm×about 28 mm. The length of the device 107 can range from about 100 mm to about 400 mm. For example, the length of the device 107 can be about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, about 310 mm, about 320 mm, about 330 mm, about 340 mm, about 350 mm, about 360 mm, about 370 mm, about 380 mm, about 390 mm, or about 400 mm.

In some embodiments, the length of the elongated body of the device 107 can range from about 100 mm to about 400 mm. For example, the length of the elongated body of the device 107 can be about 100 mm, about 110 mm, about 120 mm, about 130 mm, about 140 mm, about 150 mm, about 160 mm, about 170 mm, about 180 mm, about 190 mm, about 200 mm, about 210 mm, about 220 mm, about 230 mm, about 240 mm, about 250 mm, about 260 mm, about 270 mm, about 280 mm, about 290 mm, about 300 mm, about 310 mm, about 320 mm, about 330 mm, about 340 mm, about 350 mm, about 360 mm, about 370 mm, about 380 mm, about 390 mm, or about 400 mm.

In some embodiments, the length of the elongated body of the device 107 has a longest length of about 200 mm to about 400 mm. For example, the longest length of the elongated body of the device 107 can be about 200 mm, about 210 mm, about 220 mm, about 230 mm, about 240 mm, about 250 mm, about 260 mm, about 270 mm, about 280 mm, about 290 mm, about 300 mm, about 310 mm, about 320 mm, about 330 mm, about 340 mm, about 350 mm, about 360 mm, about 370 mm, about 380 mm, about 390 mm, or about 400 mm.

In some embodiments, the longest length of the elongated body of the device 107 is no greater than about 200 mm, no greater than about 210 mm, no greater than about 220 mm, no greater than about 230 mm, no greater than about 240 mm, no greater than about 250 mm, no greater than about 260 mm, no greater than about 270 mm, no greater than about 280 mm, no greater than about 290 mm, no greater than about 300 mm, no greater than about 310 mm, no greater than about 320 mm, no greater than about 330 mm, no greater than about 340 mm, no greater than about 350 mm, no greater than about 360 mm, no greater than about 370 mm, no greater than about 380 mm, no greater than about 390 mm or no greater than about 400 mm.

The width, or the widest width, of the device 107 can range from about 20 mm to about 30 mm. For example, the width, or the widest width, of the device 107 can be about 20 mm, about 21 mm, about 22 mm, about 23 mm, about 24 mm, about 25 mm, about 26 mm, about 27 mm, about 28 mm, about 29 mm, or about 30 mm.

The depth, or the deepest depth, of the device 107 can range from about 1 mm to about 50 mm. For example, the depth, or the deepest depth, of the device 107 can be about 1 mm, about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm

The device 107 can be shaped and sized for intraoral image acquisition, such as by insertion into a mouth of an imaging subject and passing the over one or more intraoral structures (e.g., a tooth or teeth) at a suitable distance to acquire surface data from the subject's dentition, such as teeth and gums. Non-limiting examples of the imaging device shape include a cube, a sphere, a cylinder, a square, a rectangle, and a circle. The head end 102 can be sized to fit in a human mouth.

In some embodiments, the head end 102 has a width of about 1 cm, about 2 cm, about 3 cm, about 4 cm, or about 5 cm. In some embodiments, the head end 102 has a width of no greater than about 1 cm, no greater than about 2 cm, no greater than about 3 cm, no greater than about 4 cm, or no greater than about 5 cm. In some embodiments, the head end 102 has a width of 2 cm to 3 cm.

In some embodiments, the head end 102 has a depth of about 1 cm, about 2 cm, about 3 cm, about 4 cm, or about 5 cm. In some embodiments, the head end 102 has a depth of no greater than about 1 cm, no greater than about 2 cm, no greater than about 3 cm, no greater than about 4 cm, or no greater than about 5 cm. In some embodiments, the head end 102 has a depth of 2 cm to 3 cm.

Non-limiting examples of materials that can be used in the manufacture of the imaging device 107 include polyvinyl chloride, polyethylene, polypropylene, polystyrene, polyurethane, polyethylene terephthalate, polycarbonate, silicone, and combinations thereof. Further non-limiting examples of materials that can be used in the manufacture of the device include steel, low-carbon steel, medium-carbon steel, high-carbon steel, aluminum, brass, copper, lead, magnesium, nickel, titanium, zinc, and combinations thereof. Additional non-limiting examples of materials that can be used in the manufacture of the device include copper wire, aluminum wire, XHHW wire, THWN wire, and THHN wire.

Non-limiting examples of chips that can be used in the manufacture of the imaging device include dynamic random access memory chips, microprocessors, application specific integrated circuits, digital signal processors, programmable memory chips, antennas, WiFi chips, and combinations thereof.

Non-limiting examples of semiconductors that can be used in the manufacture of the imaging device include diamond, silicon, germanium, tin, silicon carbide, selenium, tellurium, boron nitride, zinc oxide, copper (I) oxide, and combinations thereof.

In some embodiments, the mass of the device 107 can range from about 1 gram to about 1000 grams. For example, the total mass of the device 107 can be about 1 gram, about 2 grams, about 3 grams, about 4 grams, about 5 grams, about 6 grams, about 7 grams, about 8 grams, about 9 grams, about 10 grams, about 15 grams, about 20 grams, about 25 grams, about 30 grams, about 35 grams, about 40 grams, about 45 grams, about 50 grams, about 60 grams, about 70 grams, about 80 grams, about 90 grams, about 100 grams, about 110 grams, about 120 grams, about 130 grams, about 140 grams, about 150 grams, about 200 grams, about 250 grams, about 300 grams, about 350 grams, about 400 grams, about 450 grams, about 500 grams, about 550 grams, about 600 grams, about 650 grams, about 700 grams, about 750 grams, about 800 grams, about 850 grams, about 900 grams, about 950 grams, or about 1000 grams. In some embodiments, the device 107 has a total mass of less than about 100 grams.

In some embodiments, the device 107 has an average output power of about 1 μW, about 2 μW, about 3 μW, about 4 μW, about 5 μW, about 6 μW, about 7 μW, about 8 μW, about 9 μW, about 10 μW, about 20 μW, about 30 μW, about 40 μW, about 50 μW, about 60 μW, about 70 μW, about 80 μW, about 90 μW, about 100 μW, about 200 μW, about 300 μW, about 400 μW, about 500 μW, about 600 μW, about 700 μW, about 800 μW, about 900 μW, about 1 mW, about 2 mW, about 3 mW, about 4 mW, about 5 mW, about 6 mW, about 7 mW, about 8 mW, about 9 mW, about 10 mW, about 15 mW, about 20 mW, about 25 mW, about 30 mW, about 35 mW, about 40 mW, about 45 mW, about 50 mW, about 60 mW, about 70 mW, about 80 mW, about 90 mW, or about 100 mW.

In some embodiments, the device described herein can be used repeatedly without overheating. In some embodiments, the device described herein does not comprise a self-cooling mechanism. In some embodiments, the device described herein does not comprise a fan. In some embodiments, the device described herein does not comprise moving parts.

The set of 2D images taken by the head end 102 comprises a face comprising an intra-oral camera during the image acquisition process can be transmitted via the tail end 103 comprising a connection to the computational unit 104 during a data transmission process.

The tail end 103 comprising a connection can use any suitable communications link during the data transmission process. Non-limiting examples of the communications link include a wired connection or a wireless connection based upon, for example, IEEE 802.11 (also known as wireless Ethernet), BlueTooth©, or any other suitable wireless standard using, e.g., a radio frequency, infrared, ultrasound, or other wireless communication medium. Non-limiting examples of the computational unit include personal computers, such as a portable PC; slate and tablet PC, such as Apple® iPad and Samsung® Galaxy Tab; telephones; smartphones, such as Apple® iphone, Android-enabled device, Windows® Phone, and Blackberry®; smart watches, such as Apple® Watch; smart glasses, such as Google® Glass; virtual reality devices; augmented reality devices; head mounted devices, such as a VR headset; or personal digital assistants.

The computational unit 104 generates the 3D representation of a tooth or teeth from the set of 2D images obtained by the optical sensor. The computational unit 104 can also generate control signals to the intra-oral camera 102, which, in addition to image acquisition commands, can include conventional camera controls such as focus or zoom.

In some embodiments, the computational unit 104 generates a three-dimensional representation of a tooth based at least in part on the set of 2D images obtained by the optical sensor 201.

In some embodiments, the computational unit 104 generates a three-dimensional representation of a tooth based at least in part on an image of the tooth obtained by the optical sensor.

In some embodiments, the computational unit 104 generates a three-dimensional representation of a tooth based at least in part by comparing an image of the tooth obtained by the optical sensor to a reference image of a reference tooth, wherein the reference image of the reference tooth is associated with a pre-determined three-dimensional structure of the reference tooth.

In some embodiments, the computational unit 104 generates a three-dimensional representation of a tooth based at least in part by comparing an image of the tooth obtained by the optical sensor to a plurality of reference images, wherein each of the reference images is independently of a corresponding reference tooth, wherein each reference image is associated with a pre-determined three-dimensional structure of the corresponding reference tooth; and determining which reference image of the plurality is most similar to the image of the tooth obtained by the optical sensor.

The display unit 105 presents the captured 2D images or the generated 3D dental representation. The display unit 105 can include any display suitable for video or other rate rendering at a level of detail corresponding to the acquired data or a rendered version of the acquired data. Non-limiting examples of the display unit 105 include cathode ray tube displays, liquid crystal displays, light emitting diode displays, plasma displays, touch screen displays, televisions, projectors, smartphones, smart watches, tablets, and electronic glasses.

In some embodiments, the data transmission process is wireless. In some embodiments, the data transmission process can transmit data over a distance of about 1 meter, about 2 meters, about 3 meters, about 4 meters, about 5 meters, about 6 meters, about 7 meters, about 8 meters, about 9 meters, about 10 meters, about 11 meters, about 12 meters, about 13 meters, about 14 meters, about 15 meters, about 16 meters, about 17 meters, about 18 meters, about 19 meters, or about 20 meters.

The system described herein can be used by a subject hourly, daily, weekly, monthly, yearly, occasionally, frequently, continuously, or chronically. The system described herein can be used by a subject as needed based on a condition of the subject, upon a dentist's or orthodontist's recommendation, as desired by the subject, as required to monitor the condition of the subject properly, or for diagnostic or research purposes.

In some embodiments, the system described herein can be used without calibration between use. The system described herein can be calibrated after at least 2 uses, at least 3 uses, at least 4 uses, at least 5 uses, at least 6 uses, at least 7 uses, at least 8 uses, at least 9 uses, or at least 10 uses. The system described herein can be used without calibration between different subjects. The system described herein can be used without calibration between the same subject.

### Intra-Oral Camera

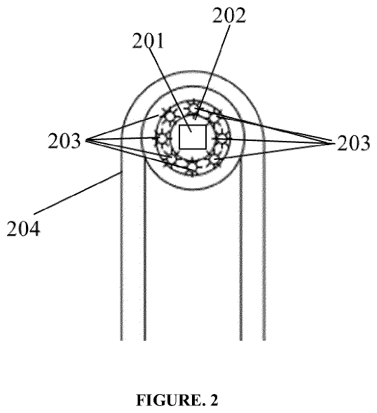

FIG. 2 illustrates the head end comprising an intra-oral camera or camera system for obtaining image(s) of a tooth during the image acquisition process. The intra-oral camera comprises at least one optical sensor 201, an optical lens 202, at least one source of illumination 203, and a camera tip 204 that encloses the sensors.

In some embodiments, the optical lens 202 is a fixed focus lens. The focal length of the fixed focus lens disclosed herein can range from about 18 mm to about 35 mm. Any suitable fixed focus lens that satisfies the focal length range can be used. For example, the focal length of the fixed focus lens can be about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, or about 35 mm.

In some embodiments, the intra-oral camera comprises a camera tip 204. In some embodiments, the camera tip 204 encloses the at least one optical sensor 201.

The optical sensor 201 can obtain an image or a set of images of a tooth by detecting light waves that pass through or reflect off the subject's tooth during the image acquisition process.

Any suitable optical sensor that detects and conveys information used to form an image can be used. Non-limiting examples of the optical sensor 201 include a charge-coupled device (CCD) sensor, an active-pixel sensor (CMOS), and a RGB sensor. In some embodiments, the frame rate of the RGB sensor can be between 5-60 hz. For example, the frame rate can be about about 5 hz, about 6 hz, about 7 hz, about 8 hz, about 9 hz, about 10 hz, about 11 hz, about 12 hz, about 13 hz, about 14 hz, about 15 hz, about 16 hz, about 17 hz, about 18 hz, about 19 hz, about 20 hz, about 21 hz, about 22 hz, about 23 hz, about 24 hz, about 25 hz, about 26 hz, about 27 hz, about 28 hz, about 29 hz, about 30 hz, about 31 hz, about 32 hz, about 33 hz, about 34 hz, about 35 hz, about 36 hz, about 37 hz, about 38 hz, about 39 hz, about 40 hz, about 41 hz, about 42 hz, about 43 hz, about 44 hz, about 45 hz, about 46 hz, about 47 hz, about 48 hz, about 49 hz, about 50 hz, about 51 hz, about 52 hz, about 53 hz, about 54 hz, about 55 hz, about 56 hz, about 57 hz, about 58 hz, about 59 hz, or about 60 hz.

Non-limiting examples of the image plane formed by the optical sensor 201 include a square, a circle, a rectangle, and an ellipse.

In some embodiments, the optical sensor 201 has a field of view, wherein the optical sensor 201 has a central optical axis that is perpendicular to the field of view, wherein the central optical axis is perpendicular to the shaft central axis, wherein the optical sensor 201 is configured to capture an image or a set of images.

In some embodiments, the optical sensor is an autofocus lens. In some embodiments, the autofocus lens is an autofocus lens with two or more positions. In some embodiments, the autofocus lens is fixed to a known position to emulate a fixed focus lens. In some embodiments, the autofocus lens emulating the fixed focus lens has a focal length ranging from about 18 mm to about 35 mm. For example, the focal length of the fixed focus lens can be about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, or about 35 mm.

In some embodiments, the tooth is an animal tooth situated in an animal mouth. In some embodiments, the tooth is a human tooth situated in a human mouth. In some embodiments, the human mouth comprises teeth neighboring the human tooth. In some embodiments, the method described herein further comprises exposing the teeth neighboring the human tooth to the optical sensor 201.

In some embodiments, the human tooth situated in an arch of a human mouth, wherein the method described herein comprises exposing all teeth of the arch to the optical sensor. In some embodiments, the method described herein comprises exposing all teeth of the human mouth to the optical sensor.

In some embodiments the method described herein comprises exposing the tooth to an optical sensor comprises inserting the optical sensor into a mouth that contains the tooth.

In some embodiments, the intra-oral camera comprises a first optical sensor and a second optical sensor. In some embodiments, the first optical sensor has a first field of view, wherein the first optical sensor has a first central axis that is perpendicular to the first field of view, wherein the first central axis is perpendicular to the shaft central axis. In some embodiments, the second optical sensor has a second field of view, wherein the second optical sensor has a second central axis that is perpendicular to the second field of view, wherein the second central axis is perpendicular to the shaft central axis, wherein the second central axis is parallel to the first central axis. In some embodiments, the first field of view and the second field of view overlap.

In some embodiments, the first optical sensor is configured to take a first two-dimensional image, and the second optical sensor is configured to take a second two-dimensional image, wherein the first two-dimensional image and the second two-dimensional image partially overlap and are not coterminous.

The sources of illumination 203 supplement illumination of the subject's tooth during the image acquisition process. Any suitable light or lighting system can be used as the sources of illumination 203. In some embodiments, the sources of illumination produce visible or near-visible radiant energy. Non-limiting examples of the sources of illumination include incandescent, fluorescent, high intensity discharge (HID), solid-state lighting (SSL), light-emitting diodes (LED), laser diodes, organic LEDS, infrared, visible light, ultraviolet (UV) light, and any other semiconductor light sources. The light or lighting system can be emitted in the form of a strobe, flash, or spotlight. In some embodiments, the sources of illumination are oriented to illuminate the field of view.

In some embodiments, the device 107 comprises heating wires. In some embodiments, the heating wires are heated to prevent fog in the intraoral camera. In some embodiments, the intraoral camera can be heated by the sources of illumination, such as LED or by the heating wires. In some embodiments, the heating can prevent foggy images as the oral cavity is typically humid and hotter than the room temperature.

In some embodiments, the optical sensor and the sources of illumination are the same. In some embodiments, the optical sensor and the sources of illumination are different.

In some embodiments, the intra-oral camera comprises at least one source of illumination. In some embodiments, the intra-oral camera comprises a plurality of sources of illumination. In some embodiments, the intra-oral camera comprises at least one, at least two, at least three, at least four, at least five, at least six, at least seven, at least eight, at least nine, or at least ten sources of illumination.

In some embodiments, the intra-oral camera further comprises a shutter. In some embodiments, the shutter is a rolling shutter. In some embodiments, the rolling shutter is a mechanical rolling shutter or an electronic rolling shutter. In some embodiments, the shutter is not a global shutter.

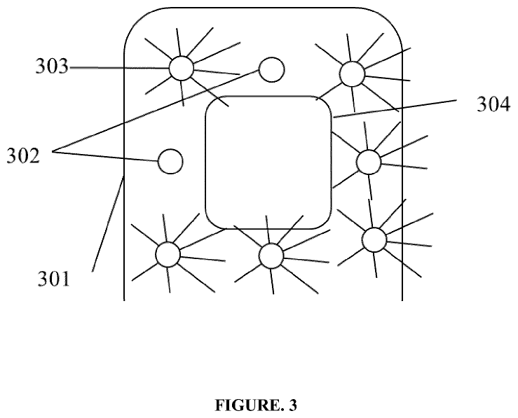

FIG. 3 illustrates an embodiment of the intra-oral camera or camera system described herein. The intra-oral camera can comprise a plurality of sources of illumination 302 303. The sources of illumination 302 303 can be arranged in a pseudo-random pattern around the optical sensor 304. During the image acquisition process, one or more sources of illumination can be turned on 303 or turned off 302 at any time. A combination of the sources of illumination can be turned on 303 or off 302 simultaneously during the image acquisition process. Starting and stopping of the sources of illumination can be controlled by the contour 108 illustrated in FIG. 1. The optical sensor can be enclosed in a camera tip 301.

In some embodiments, described herein is a method of the image acquisition process described herein. The method comprises (i) exposing a subject's tooth to an optical sensor 401 and (ii) obtaining by the optical sensor 401 a set of two-dimensional images of a tooth. During the image acquisition process, the tooth 405 is placed within the focal length 403 of the fixed focus lens 406 of the optical sensor 401 described herein.

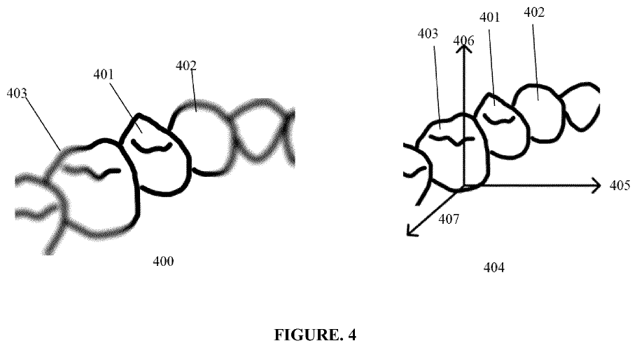

FIG. 4 illustrates an embodiment of recovering the scale of a captured 2D image 400 using the method described herein. The tooth 401 is within the focal length (i.e., 18-36 mm) of the fixed focus lens, the tooth appears sharp in the captured 2D image. The tooth 402 is placed in a position longer than the focal length of the fixed focus lens, the tooth 402 appears more blurry and darker than tooth 401 captured from within the focal length in the captured 2D image. The tooth 403 is placed in a position shorter than the focal length of the fixed focus lens, the tooth 403 appears more blurry and brighter than tooth 401 captured from within the focal length in the captured 2D image. In some embodiments, the brightness of the tooth or teeth in the captured 2D image can be used to determine the scale and depth of the tooth or teeth. In some embodiments, the shape of the shadows casted by the tooth or teeth in the captured 2D image can be used to determine the scale and depth of the tooth or teeth. 404 illustrates the recovered scale of the teeth 401, 402, 403 on the x-axis 405, y-axis 406, and z-axis 407.

In some embodiments, the way the lens renders out-of-focus points of light (i.e., bokeh) can be used to determine the scale of the tooth or teeth in the captured 2D image. In some embodiments, the scale of the teeth 401, 402, 403 are recovered by calibration of relationship between the distance, blurriness and characteristics of the bokeh in the captured 2D images. In some embodiments, the scale of the teeth 401, 402, 403 is recovered using a second camera or additional calibrated pattern, such as the pattern and size of reflections of the LED lights, that is added to the scene.

### Trained, Machine-Learning Model

In some embodiments, the computational unit 104 comprises an algorithm. The algorithm can be a trained, machine-learning model.

In some embodiments, a method described herein comprises: (i) exposing a tooth to an optical sensor; (ii) obtaining by the optical sensor a set of two-dimensional images of a tooth; and (iii) generating a three-dimensional representation of the tooth based at least in part on the set of two-dimensional images of the tooth. In some embodiments, generating the three-dimensional representation of the tooth is based at least in part on the set of two-dimensional images of the tooth comprises comparing the set of two-dimensional images of the tooth to a reference image of a reference tooth. In some embodiments, the reference image of the reference tooth is associated with a pre-determined, three-dimensional structure of the reference tooth. In some embodiments, the method described further comprises adopting the pre-determined, three-dimensional structure of the reference tooth as the three-dimensional representation of the tooth. In some embodiments, the set of two-dimensional images of the tooth and the reference image of a reference tooth are taken by the same optical sensor. In some embodiments, the set of two-dimensional images of the tooth and the reference image of a reference tooth are taken by different optical sensor.

In some embodiments, the generating the three-dimensional representation of the tooth based at least in part on the set of two-dimensional images of the tooth comprises: (i) comparing the set of two-dimensional images of the tooth to a plurality of reference images, wherein each of the reference images is independently of a corresponding reference tooth, wherein each reference image is associated with a pre-determined, three-dimensional structure of the corresponding reference tooth; (ii) determining which reference image of the plurality is most similar to the set of two-dimensional images of the tooth; and (iii) adopting as the three-dimensional representation of the tooth the pre-determined, three-dimensional structure of the reference tooth associated with the reference image that is most similar to the set of two-dimensional images of the tooth. In some embodiments, the comparing the set of two-dimensional images of the tooth to the plurality of reference images comprises using a trained, machine-learning model to match a portion of the two-dimensional images of the tooth with a portion of a reference image, wherein the trained, machine-learning model is trained on at least a subset of the plurality of reference images.

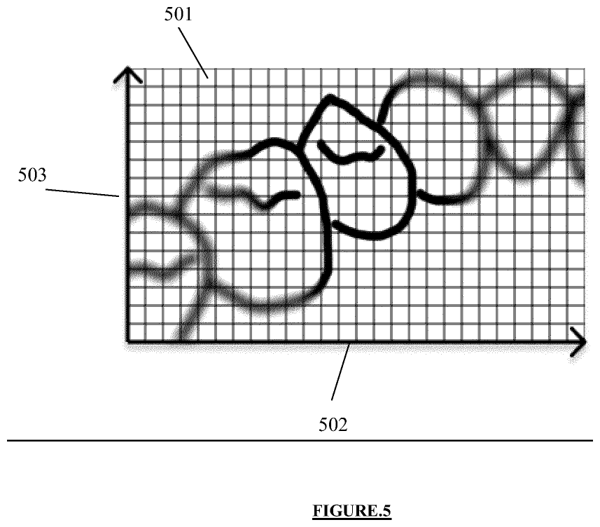

In some embodiments, the input for the trained, machine-learning model is the set of 2D images in pixels. FIG. 5 illustrates an embodiment of a 2D image in pixels. 501 represents an individual pixel. The 2D image comprises an x-axis 502 and a y-axis 503.

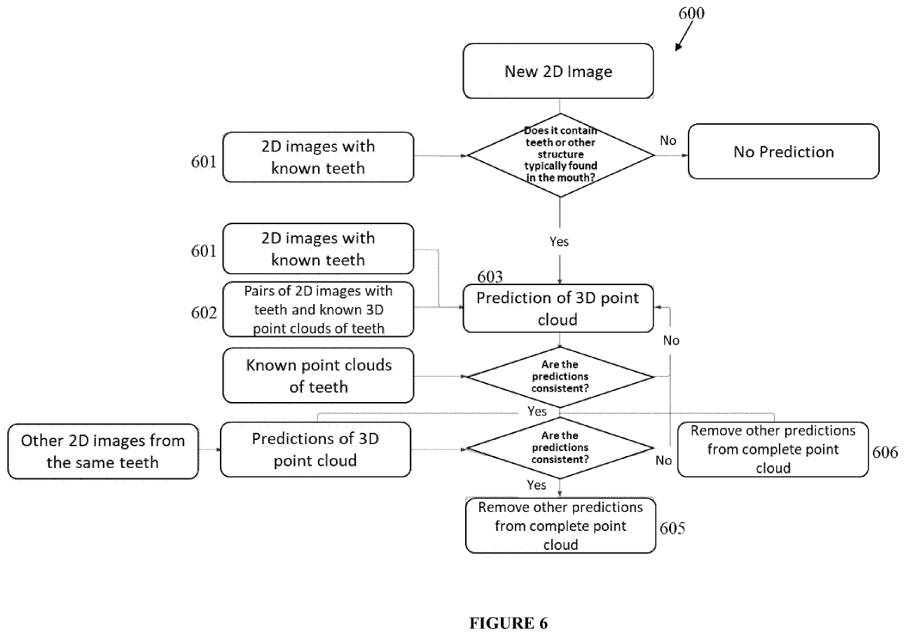

FIG. 6 illustrates an example of a schematic of a workflow 600 for the trained, machine-learning model described herein.

The workflow 600 comprises a training set, the training set comprising: (1) a plurality of references images, wherein each of the reference images is independently of a corresponding reference tooth 601; and (2) pairs of plurality of reference images, wherein each reference image is associated with a pre-determined, 3D structure of the corresponding reference tooth 602. In some embodiments, the pre-determined 3D structure of the corresponding reference tooth is in the form of 3D point clouds or 3D mesh.

The trained, machine-learning model can compare the set of 2D images obtained by the optical sensor to the plurality of references images 601 and determine a subset of 2D images in the set of 2D images obtained by the optical sensor that contain a tooth or teeth.

The trained, machine-learning model can then compare the subset of 2D images with the plurality of references images 601 and the pairs of plurality of reference images 602 and generate a prediction 3D point cloud 603. The prediction 3D point cloud 603 then undergoes a comparison with pre-determined, three-dimensional structure of the reference tooth in 602, by which the trained, machine-learning algorithm generates a subset of consistent predictions 604. The workflow 600 is repeated until a final subset of predictions 605 is generated to complete point cloud.

In some embodiments, a computer code or manual adjustments are used to remove or filter out 606 2D images not containing teeth or other structure typically found in the mouth and to remove or filter out 606 non-consistent predictions, thereby producing the filtered motion predictions to complete point clouds as shown.

In some embodiments, the trained, machine-learning model is trained on at least a subset of the plurality of reference images.

In some embodiments, the trained, machine-learning model can be used to determine the scale or depth of the subject by the brightness of the obtained 2D image, pattern or sizes of the reflections of the sources of illumination in the obtained 2D image, characteristics of the tooth in the obtained 2D image, shapes of shadows in the obtained 2D image, or a combination thereof. In some embodiments, machine-learning algorithm can determine the scale of the subject by calibration of relationship between the distance and blurriness of the obtained 2D images.

In some embodiments, the trained, machine-learning algorithm comprises a training set, wherein a first portion of the training set is used for training a predictive model, and a second portion of the training set is used to validate the predictive model by testing the model's predictive ability. The manner in which the first portion and the second portion of the training set are chosen is not limited. The first portion and the second portion can be chosen dynamically for training and validation.

In some embodiments, the training set comprises a plurality of references images. The reference images can have a plurality of features associated therewith, whereby each training sample can be represented by a feature vector (generated on the basis of the plurality of features) and an associated label. The feature vector can be a list of features, each feature of the feature vector being a measurable property of the reference images. The label can correspond to an output of the reference images, i.e. a classification or a prediction that is desirable to know.

In some embodiments, the prediction is the depth on a per-pixel basis. In some embodiments, the trained, machine learning model is trained to predict the depth given the reference images

In some embodiments, each of the reference images is independently of a corresponding reference tooth. In some embodiments, each reference image is associated with a pre-determined, 3D structure of the corresponding reference tooth.

In some embodiments, the training set described herein can be any form of an electronic file or a document that can be stored on a computer readable medium such as, but not limited to, the solid-state drive. The training set can include any type of media. Non-limiting examples of media include a text file, an HTML page, a PDF document, a formatting information, a metadata, an audio recording, an image, and a video recording.

Non-limiting examples of features vectors include query-independent (i.e. static features), query-dependent (i.e. dynamic features), and query level features. Non-limiting examples of features include TF, TF-IDF, BM25, IDF sums and lengths of document's zones and document's PageRank, HITS ranks, and other variants.

Systems and methods described herein can be implemented by way of a machine-or computer-executable code or software stored on an electronic storage location of the server, such as, for example, on a memory or electronic storage unit. During use, the code can be executed by a processor.

All or portions of the trained, machine-learning model can at times be communicated through the Internet or various other telecommunications networks. Such communications can support loading of the algorithm from one computer or processor into another, for example, from a management server or host computer into the computer platform of an application server. Another type of media that can bear the algorithm elements includes optical, electrical, and electromagnetic waves, such as those used across physical interfaces between local devices, through wired and optical landline networks, and over various air-links. The physical elements that carry such waves, such as wired or wireless links, or optical links, also can be considered as media bearing the software.

A machine-readable medium, incorporating computer-executable code, can take many forms, including a tangible storage medium, a carrier wave medium, and physical transmission medium. Non-limiting examples of non-volatile storage media include optical disks and magnetic disks, such as any of the storage devices in any computer, such as can be used to implement the algorithm. Volatile storage media include dynamic memory, such as a main memory of such a computer platform. Tangible transmission media include coaxial cables, copper wire, and fiber optics, including wires that comprise a bus within a computer system. Carrier-wave transmission media can take the form of electric or electromagnetic signals, or acoustic or light waves such as those generated during radio frequency (RF) and infrared (IR) data communications.

Forms of computer-readable media include: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, and any other medium from which a computer can read programming code or data. Many of these forms of computer readable media can be involved in carrying one or more sequences of one or more instructions to a processor for execution.

Methods or systems described herein can be facilitated with the aid of applications, or apps, that can be installed on an electronic device of the subject. An app can include a graphical user interface (GUI) on a display of the devices or systems described herein. The app can be programmed or otherwise configured to perform various functions of the system, such as, for example, permitting a subject to manage, such as create and edit, the 3D representation. GUIs of apps can display on an electronic device of the subject. Non-limiting examples of electronic devices include computers, televisions, smartphones, tablets, and smart watches. The electronic device can include, for example, a passive screen, a capacitive touch screen, or a resistive touch screen. The electronic device can include a network interface and a browser that allows the subject to access various sites or locations, such as web sites, on an intranet or the Internet. The app is configured to allow the mobile device to communicate with a server.

In some embodiments, the disclosure provides a kit comprising: (i) the device described herein; and (ii) written instructions describing a use of the device in scanning a tooth.

In some embodiments, the kit comprises (i) the device described herein and (ii) written instructions describing a use of the device described herein in the method described herein.

1-51. (canceled)

52. A system for generating a three-dimensional representation of a user's dental feature, comprising a device having an elongated body comprising:
a) a head end comprising a face;
b) a tail end comprising a wireless electrical connection to a power supply;
c) a shaft connecting the head end and the tail end;
wherein:
the shaft comprises a shaft central axis through the shaft from the head end to the tail end;
the face comprises:
at least one optical sensor having a field of view, wherein the optical sensor has a central optical axis that is perpendicular to the field of view, wherein the central optical axis is perpendicular to the shaft central axis, and wherein the optical sensor is configured to capture a bidimensional image of the dental feature and comprises a fixed focus lens; and
at least one source of illumination oriented to illuminate the field of view;
the system further comprises a computational unit configured to communicate with the device, to receive the bidimensional image and to generate the three-dimensional representation of the dental feature based on the bidimensional image; and
a scale of the dental feature is determined on a basis of:
a brightness of the dental feature in the bidimensional image,
a shape of a shadow casted by the dental feature in the bidimensional image, and
a bokeh generated by the fixed focus lens in the bidimensional image.

53. The system of claim 52, wherein the face further comprises an additional optical sensor.

54. The system of claim 52, wherein the fixed focus lens is an autofocus lens emulating a fixed focus lens.

55. : The system of claim 52, wherein the optical sensor is a RGB sensor.

56. The system of claim 55, wherein the RGB sensor has a frame rate of at least 5 hz and at most 60 hz.

57. The system of claim 52, wherein the source of illumination is a light emitting diode (LED).

58. The system of claim 57, wherein the scale of the dental feature is determined also on the basis of a pattern and size of reflections of the LED.

59. A method for generating a three-dimensional representation of a user's dental feature using the system of claim 52, comprising:
a) obtaining by the optical sensor a set of bidimensional images of the dental feature; and
b) generating the three-dimensional representation of the dental feature based at least in part on the set of bidimensional images of the dental feature.

60. The method of claim 59, wherein the generating the three-dimensional representation of the dental feature comprises comparing the set of bidimensional images of the dental feature to a reference image of a reference dental feature, the reference image of the reference dental feature being associated with a pre-determined, three-dimensional structure of the reference dental feature.

61. The method of claim 60, wherein the reference image of the reference dental feature is taken by the optical sensor.

62. The method of claim 60, wherein the reference image of the reference dental feature is synthetically generated.

63. The method of claim 60, further comprising adopting the pre-determined, three-dimensional structure of the reference dental feature as the three-dimensional representation of the dental feature.

64. The method of claim 59, wherein the generating the three-dimensional representation of the dental feature comprises:
1) comparing the set of bidimensional images of the dental feature to a plurality of reference images, wherein each of the reference images is independently of a corresponding reference dental feature, wherein each reference image is associated with a pre-determined, three-dimensional structure of the corresponding reference dental feature; and
2) determining which reference image of the plurality is most similar to the set of bidimensional images of the dental feature.

65. The method of claim 64, further comprising:
3) adopting as the three-dimensional representation of the dental feature the pre-determined, three-dimensional structure of the reference dental feature associated with the reference image that is most similar to the set of bidimensional images of the dental feature.

66. The method of claim 64, wherein the comparing the set of bidimensional images of the dental feature to the plurality of reference images comprises using a trained, machine-learning model to match a portion of the bidimensional images of the dental feature with a portion of a reference image, wherein the trained, machine-learning model is trained on at least a subset of the plurality of reference images.

67. The method of claim 59, wherein the set of bidimensional images is three images of the dental feature, in particular comprising a buccal image of the dental feature, an occlusal image of the dental feature, and a lingual image of the dental feature.

68. The method of claim 59, wherein in the set of bidimensional images, each bidimensional image overlaps with at least one other bidimensional image, in particular by at least about 10%.

69. The method of claim 59, wherein the three-dimensional representation of the dental feature is a 3D point cloud.
